(19) United States
(12) Patent Application Publication
(10) Pub. No.: US 2003/0077818 A1
(43) Pub. Date: Apr. 24, 2003

(54) Compositions and methods for targeting interleukin-12 to malignant endothelium

(76) Inventors: Erin B. Dickerson, Madison, WI (US); Stuart C. Helfand, Madison, WI (US)

Correspondence Address: LICATLA & TYRRELL P.C., 66 E. MAIN STREET, MARLTON, NJ, 08053, US

(21) Appl. No.: 09/801,485

(22) Filed: Mar. 8, 2001

(51) Int. Cl. A61K 38/20; C12N 5/06; C07H 21/04

(52) U.S. Cl. 435/325; 536/23.5; 424/85.2; 435/320.1

(57) ABSTRACT

Fusion proteins containing a mammalian interleukin-12 operably linked to a RGD-containing peptide as well as nucleic acid sequences, vectors and host cells for expression of these fusion proteins are provided. Also provided are method of using these fusion proteins to inhibit tumor growth and to decrease the toxicity associated with interleukin-12 administration.

### INTRODUCTION

Work presented herein was supported by the National Institutes of Health (Grant No. CA86264) and the U.S. Federal Government may have certain rights in this invention.

### FIELD OF THE INVENTION

The present invention relates to bifunctional fusion proteins that simultaneously target vascular and immune compartments within the tumor environment. Fusion proteins of the present invention comprise a ligand for αᵥβ₃, preferably a small peptide sequence comprising the sequence arginine-glycine-aspartic acid (RGD), that specifically directs the fusion protein to angiogenic endothelial cells and αᵥβ₃ positive tumor cells. This vascular targeting peptide is coupled to interleukin-12 (IL-12), a cytokine with antiangiogenic activity mediated by induction of interferon-γ and other antiangiogenic chemokines. Accordingly, using a fusion protein of the present invention, high concentrations of IL-12 can be targeted to the tumor microenvironment thereby activating the tumoricidal responses of immune cells in situ and decreasing toxic side effects relating to the activity of IL-12 on nontumor tissues.

### BACKGROUND OF THE INVENTION

The ability of cancer cells to metastasize correlates well with their capacity to initiate angiogenesis, the formation of new blood vessels within tumor tissue (Folkman, J. J. Natl Cancer Inst. 1990 82:4-6; Folkman, J. Nature Med. 1995 1:27-31; and Liotta et al. Cell 1991 64:327-336). The process of angiogenesis can be inhibited by a number of substances including retinoids, vitamin D, TGF-β, interferons-γ and -α, interleukin-1β, fumagillin and its derivatives AGM-1470 and angiostatin.

Recently interleukin-12 (IL-12) has been reported to have antiangiogenic properties mediated through the induction of IFN-γ and other downstream proteins that this cytokine induces (Angiolillo et al. Ann. NY Acad. Sci. 1996 795:158-167; Dias et al. Int. J. Cancer 1998 75:151-157; and Sgadari et al. Blood 1997 89:2635-2643). IL-12 exhibits a number of activities potentially important in cancer therapy. In humans and mice, IL-12 is a potent activator of natural killer (NK) cell activity (Kobayashi et al. J. Exp. Med. 1989 170:827-845) and a major inducer of IFN-γ from NK and T lymphocytes (Chan et al. J. Exp. Med. 1991 173:869-879), a cytokine with important immune cell activating capabilities. IFN-γ is also an essential mediator of the antiangiogenic effects ascribed to IL-12 (Voest et al. J. Natl Cancer Inst. 1995 87:581-586; Majewski et al. J. Invest. Dermatol. 1996 106:1114-1118). IL-12 enhances tumor cell killing mediated by immune cells specifically directed toward tumor targets by antitumor antibodies (antibody-dependent cellular cytotoxicity, ADCC) (Lieberman et al. J. Surg. Res. 1991 50:410-415). IL-12 stimulates nitric oxide production in vivo resulting in delayed tumor progression in mice (Wigginton et al. 1996 Cancer Res. 56:1131-1136). Endogenous IL-12 production has also been documented to gradually diminish as tumor burden increases (Handel-Fernandez et al. J. Immunol. 1997 158:280-286), thus forming a rationale for providing IL-12 to cancer patients to reconstitute cell-mediated antitumor responses. IL-12 is also a potent inhibitor of tumor-driven angiogenesis (Voest et al. J. Natl Cancer Inst. 1995 87:581-586; Majewski et al. J. Invest. Dermatol. 1996 106:1114-1118) demonstrating significant in vivo inhibition of tumor blood vessel formation in mice mediated through IFN-γ inducible protein-10 (IP-10; Sgadari et al. Blood 1996 87:3877-3882), a chemokine that has a potent antiangiogenic effect on the vasculature of growing tumors (Angiolillo et al. Ann NY Acad. Sci. 1996 795:158-167; Arenberg et al. J. Exp. Med. 1996 184:981-992). In vitro, it inhibits the formation of tube-like structures by endothelial cells (Angiolillo et al. J. Exp. Med. 1995 182:155-162). In vivo, induction of IP-10 by IL-12 results in central tumor necrosis with surrounding blood vessels showing intimal thickening, endothelial cell apoptosis, and partial to complete occlusion of the vessel lumens by thrombosis (Angiolillo et al. Ann NY Acad. Sci. 1996 795:158-167, Dias et al. Int. J. Cancer 1998 75:151-157). IP-10 is a chemoattractant for T cells and monocytes, supporting a role for it in leukocyte recruitment (Luster, A. D. and Leder, P. J. Exp. Med. 1993 178:1057-1065; Taub et al. J. Exp. Med. 1993 177:1809-1814). More recently, IL-12 has been shown to exert antiangiogenic effects through its role as a regulator of VEGF and matrix metalloproteinase (MMP) production (Dias et al. Int. J. Cancer 1998 78:361-365). In addition, IL-12 has been shown to synergize with IL-2 to exert an antiangiogenic effect (Watanabe et al. Am. J. Pathol. 1997 150:1869-1880), thereby depriving growing tumors of essential blood supply (Auerbach, W. and Auerbach, R. Pharmac. Ther. 1994 63:263-311; Gasparini, G. Crit. Rev. Onc. Hematol. 1997 26:147-162).

In preclinical studies, recombinant IL-12 was shown to mediate destruction of established tumors in mice (Dias et al. Int. J. Cancer 1998 75:151-157; Brunda et al. J. Exp. Med. 1993 178:1223-1230; Nastala et al. J. Immunol. 1994 153:1697-1706; Zou et al. Intl Immunol. 1995 7:1135-1145; and O'Toole et al. J. Immunol. 1993 150:294) and also to exert an antimetastatic effect, especially when combined with IL-2 (Wigginton et al. Cancer Res. 1996 56:1131-1136).

However, like many cancer therapies, a major obstacle to IL-12 therapy has been its appreciable toxicity, including death, in humans (Soiffer et al. Blood 1993 82:2790-2796; Atkins et al. Clin. Cancer Res. 1997 3:409-417; Leonard et al. Blood 1997 90:2541-2548; and Robertson et al. Clin. Cancer Res. 1999 5:9-16). Attempts have been made to alter the dosing regime to downregulate the extreme and life threatening systemic IFN-γ peak that follows multi-day repetitive IL-12 dosing in humans (Atkins et al. Clin. Cancer Res. 1997 3:409-417; Leonard et al. Blood 1997 90:2541-2548). While better tolerated clinically, however, this altered dosing regime results in inferior tumor control in mice (Coughlin et al. Cancer Res. 1997 57:2460-2467).

Various approaches for reducing toxicity by targeting anticancer agents to the tumor tissue have been described. In one embodiment, a chemotherapeutic drug is linked to a ligand specific for a binding partner expressed only on the surface of tumor cells. Examples of such ligands include monoclonal antibodies and peptides.

For example, the expression of αᵥβ₃ in tumor bearing animals has been shown to be a specific marker for tumor neovasculature and the dependence of angiogenic endothelium on αᵥβ₃ comprising tumor vasculature have combined to make αᵥβ₃ an important marker for cancer therapy. Targeting of αᵥβ₃ on tumor vasculature has been accomplished using antagonists such as monoclonal antibody LM609 (Brooks et al. Cell 1994 79:1157-1164) or peptides with αᵥβ₃ binding specificity such as RGD (Arap et al. Science 1998 279:377-380). In addition, a combination of an antitumor antibody-IL-2 fusion protein plus an αᵥβ₃ antagonist has proven better than either monotherapy in controlling murine syngeneic melanoma, colon carcinoma and neuroblastoma (Lode et al. Proc. Natl Acad. Sci. USA 1999 96:1591-1596).

Further, short peptides containing the RGD sequence have been shown to inhibit in vitro tumor cell invasion and in vivo tumor dissemination (Ruoslahti, E. Br. J. Cancer 1992 66:239-242). One RGD peptide containing 4 cysteines (i.e. ACDCRGDCFCG (SEQ ID NO:1) was shown to be particularly potent at inhibiting αᵥβ₃-mediated cell attachment to vitronectin (Koivunen et al. Biotechnology 1995 13:265-270). A truncated form of this peptide, specifically, CDCRGDCFC (SEQ ID NO:2) has been shown to be specific in homing to vasculature of various tumors including carcinoma, sarcoma and melanoma (Arap, W. Science 1998 279:377-380; Koivunen et al. Biotechnology 1995 13:265-270).

WO 2000/47228 discloses a chemotherapeutic comprising an angiogenesis inhibiting agent, preferably an αᵥβ₃ antagonist such as a RGD containing peptide, an antibody having antigen binding specificity for αᵥβ₃ or the βᵥβ₃ receptor, or an αᵥβ₃ mimetic, and an anti-tumor immunotherapeutic agent with a cell-effector component, preferably IL-2, and a tumor associated antigen targeting component.

In the present invention, RGD containing peptides are used to target IL-12 to angiogenic endothelial cells and αᵥβ₃ positive tumor cells.

### SUMMARY OF THE INVENTION

An object of the present invention is to provide fusion proteins comprising interleukin-12 operably linked to a RGD-containing peptide.

Another object of the present invention is to provide nucleic acid sequences, vectors and host cells expressing a fusion protein comprising interleukin-12 operably linked to a RGD-containing peptide.

Another object of the present invention is to provide a method for inhibiting growth of αᵥβ₃ positive tumor cells comprising targeting angiogenic endothelium as well as tumor cells themselves with a fusion protein comprising interleukin-12 operably linked to a RGD-containing peptide.

Another object of the present invention is to provide a method for decreasing toxic side effects associated with interleukin-12 administration in a mammal which comprises operatively linking interleukin-12 to a RGD-containing peptide via expression of a fusion protein comprising interleukin-12 and a RGD-containing peptide and administering the fusion protein to the patient.

Yet another object of the present invention is to provide a method of treating cancer in a mammal comprising administering to the mammal a fusion protein comprising interleukin-12 operably linked to an RGD-containing peptide.

### DETAILED DESCRIPTION OF THE INVENTION

The present invention relates to bifunctional fusion proteins which target αᵥβ₃ expressed on the neovasculature of tumors and αᵥβ₃ positive tumor cells. The bifunctional fusion proteins of the present invention comprise the antiangiogenic cytokine interleukin-12 (IL-12) or a biologically active fragment or variant thereof operably linked to a vascular homing peptide comprising a small peptide arginine-glycine-aspartate (RGD) with the ability to inhibit proliferation of cancer cells expressing αᵥβ₃, and to inhibit growth and development of angiogenic αᵥβ₃-expresssing endothelial cells serving the tumors.

For purposes of the present invention, by “vascular homing peptide”, it is meant, a peptide comprising the amino acid sequence arginine-glycine-aspartic acid (RGD), also referred to herein as a “RGD containing peptide”. In simplest form, this peptide may contain only the amino acids RGD. However, this peptide may further comprise additional amino acids which do not interfere with, and preferably enhance, the ability of the peptide to target αᵥβ₃. In a preferred embodiment, the vascular homing peptide comprises the amino acid sequence CDCRGDCFC (SEQ ID NO:2), also referred to herein as RGD-4C.

By “biologically active fragment or variant thereof”, it is meant fragments or variants of a full length mammalian IL-12 protein which exhibit the same or similar antiangiogenic activity of the full length IL-12 protein. By “variant” it is meant a polypeptide which differs in amino acid sequence from IL-12 by one or more substitutions, additions, deletions, fusions or truncations, or any combination thereof, but which exhibits similar activity to IL-12.

By “operably linked” it is meant that the IL-12 is attached to the vascular homing peptide in a manner which maintains both the biological activity of IL-12 and the targeting ability of the vascular homing peptide for αᵥβ₃. For example, in one embodiment, RGD-4C is linked to the carboxyterminus of the p35 or p40 subunit of the antiangiogenic cytokine IL-12. Alternatively, the RGD-containing peptide can be linked to an N-terminus of either subunit of a mammalian IL-12. Further, as will be understood by one of skill in the art upon reading this disclosure, the RGD containing peptide sequence and IL-12 can be linked at other positions in each of the sequences so long as the biological activity of IL-12 and the targeting ability of the RGD containing peptide for αᵥβ₃ are maintained. For example, since IL-12 is a heterodimer, the RGD containing peptide can also be placed on the C-terminal ends of the leader sequences which are present on the N-termini of the p35 and p40 subunits. Maintenance of these activities and abilities can be ascertained routinely by those of skill in the art in accordance with the methods described herein.

The present invention also relates to nucleic acid sequences encoding bifunctional fusion proteins comprising mammalian IL-12 or a biologically active fragment or variant thereof operably linked to a vascular homing peptide, as well as vectors and host cells comprising these nucleic acid sequences which encode the bifunctional fusion proteins. Nucleic acid sequences for IL-12 from various mammals including, but not limited to, mouse, rat, woodchuck, dog, goat, sheep, red deer and human, are described in Genbank. Such sequences can be ligated to a nucleic acid sequence encoding a RGD containing peptide routinely in accordance with well known methods. Alternatively, for shorter peptides such as those comprising only RGD, incorporation can by done by polymerase chain reaction (PCR).

In one embodiment for ligation of RGD-4C (SEQ ID NO:2) to a mouse IL-12 p35 subunit, the p35 subunit of mouse IL-12 was first isolated from cDNA reverse transcribed from RNA extracted from the spleens of 8-12 week old female FVB mice. RNA was extracted using TRIzol (Life Technologies, Grand Island, N.Y.) in accordance with the manufacturer's specifications. Purity of the RNA was increased using RNeasy spin columns (Qiagen, Valencia, Calif.). The RNA was reverse transcribed using a Superscript II RT-PCR kit (Life Technologies) in accordance with the manufacturer's specifications. The p35 subunit of murine IL-12 was isolated from the resulting cDNA using the sense primer 5′-CCGGTACCATGTGTCAATCACGTCTAC-3′ (SEQ ID NO:3) and the antisense primer 5′-CCGATATCTCAGGCGGAGCTCAGATA-3′ (SEQ ID NO:4). The restriction sites, KpnI and EcoRV, were incorporated into the 5′- and 3′-ends of the p35 sequence, respectively, following 35 rounds of PCR amplification. The PCR product was cut with restriction enzymes, gel purified and ligated into the vector pSP72 (Promega, Madison, Wis.), that has also been cut with KpnI and EcoRV and gel purified. E. coli strain JM109 cells were transfected with the vector, the plasmid purified from colonies selected on ampicillin plates, and checked for the presence of the p35 insert.

Two oligonucleotides encoding the forward and reverse DNA sequences were then synthesized (Operon Technologies, Alameda, Calif.). The sequence for the sense oligonucleotide was: 5′-CCGGGGAGCTCTGTGACTGTCGAGGCGACTGTTTTTGTTAAGATATCGG-3′ (SEQ ID NO:5); the sequence for the antisense oligonucleotide was complementary. A SacI site is present 5 bp from the end of the murine p35 cDNA sequence and this site was used for ligation of the RGD-4C DNA sequence to p35. In this procedure, the sense and antisense oligonucleotides were mixed, heated to 80° C. and rapidly cooled to allow annealing of the two strands. The homing peptide DNA was then cut with SacI and EcoRV. The pSP72 vector containing the p35 sequence was cut with the same restriction enzymes. The homing peptide sequence was ligated to the murine p35 sequence via the SacI site and to the pSP72 vector via the EcoRV site. The DNA was then transformed into competent JM109 cells. The p35RGD-4C sequence was then subcloned into the mammalian expression vector pcDNA3.1/Hydro+.

The pcDNA3.1/Hygro+ plasmid containing the p35RGD-4C was then transfected into CHO cells expressing a His tagged p40 subunit of IL-12. These CHO cells were prepared as follows. The p40 subunit of murine IL-12 was first amplified by PCR from a vector comprising the murine p40 and p35 subunits. The restriction enzyme sites KpnI and EcoRV were incorporated into the 5′- and 3′-ends of the PCR product, respectively. The primer sequence for the p40 sense strand was 5′-CCGGTACCATGTGTCCTCAGAAGCTA-3′ (SEQ ID NO:6) and the primer sequence for the antisense strand was 5′-CCGATATCCTAGGATCGGACCCTGCA-3′ (SEQ ID NO:7). The product was amplified from the vector by 35 rounds of PCR with an annealing temperature of 60° C. The PCR product was cut with the restriction enzymes KpnI and EcoRV and ligated into the vector pcDNA3.1/myc-HisA (Invitrogen, Carlsbad, Calif.), which had also been cut with the same restriction enzymes. E. coli strain JM109 was then transformed with the vector and the resulting colonies checked for the presence of the plasmid containing the p40 cDNA insert. Endotoxin-free plasmid was then prepared using an Endofree Plasmid Maxi Kit (Qiagen) according to the manufacturer's instructions. CHO cells were transfected with the endotoxin-free plasmid using Superfect (Qiagen) as indicated by the manufacturer. Cells were allowed to grow in the presence of G418 for one week, and then single cells were isolated by limiting dilution. Supernatants were tested for the presence of p40 by ELISA (Pharmingen, San Diego, Calif.), and the 10 highest producing clones from each plate were expanded. These clones were tested for the production of His-tagged p40 over 24 hours by ELISA.

Clones producing the greatest amounts, C2 and C4, were then transfected with the vector pcDNA3.1/Hydro+ containing the p35 subunit of IL-12 linked to RGD-4C. Transfected cells were allowed to grow for 2 weeks in 60 mm tissue culture dishes in the presence of 300 μg/ml hygromycin and 200 μg/ml G418. Supernatants from the transfected population were tested for the presence of the murine recombinant IL-12-vascular targeting peptide fusion protein (mrIL-12vp) using a commercially available ELISA kit for the murine p70 whole IL-12 protein (Pharmingen, San Diego, Calif.). Supernatants from CHO cells expressing only the p40 molecule and supernatants from nontransfected CHO cells were used as negative controls to test for the presence of mrIL-12vp.

The murine p35 subunit gene sequence comprises a SacI site located 5 bp from the 3′-end and methods for ligation exemplified supra for fusion proteins comprising murine IL-12 involve this restriction site. However, this site does not exist in all species. For example, neither human nor canine IL-12 contain this restriction site. Accordingly, alternative means for ligation may be required for other species.

For example, to produce fusion proteins comprising human or canine IL-12, 6 bp encoding a KpnI restriction site can be incorporated in the sequence between the end of the p35 gene and the beginning of a RGD containing peptide such as RGD-4C. This translates into an insertion of two amino acids, glycine and threonine, at the end of the p35 subunit. The sequence at the C-terminal end of the human and canine p35 subunits ligated to RGD-4C as compared to the murine p35 subunit are as follows:

Human: MSYLNASGTCDCRGDCFC (SEQ ID NO:8)

Canine: MSYLNSSGTCDCRGDCFC (SEQ ID NO:9)

Murine: MGYLSSACDCRGDCFC (SEQ ID NO:10)

The additional amino acids are in bold type and the RGD-4C homing peptide is underlined.

Thus, in one embodiment for ligation of RGD-4C to human IL-12, RNA of the human p40 subunit is first extracted using TRIzol reagent (Life Technologies, Gaithersburg, Md.) from peripheral blood lymphocytes (PBL) that have been stimulated for 40 hours with 0.0075% fixed SAC cells (Pansorbin, Calbiochem, La Jolla, Calif.). Reverse transcription can be carried out according to the manufacturer's instructions using the Superscript Preamplification System (Life Technologies, Inc.). The restriction enzyme sites for KpnI and EcoRV can be incorporated into the 5′- and 3′-ends of the PCR product, respectively, during the PCR amplification reaction. In this embodiment, the primer sequence for the p40 sense strand is 5′-CCGGTACCATGTGTCACCAGCAGTTG-3′ (SEQ ID NO:11) and the primer sequence for the antisense strand is 5′-CCGATATCCTAACTGCAGGGCACAGA-3′ (SEQ ID NO:12). The product can be amplified by 35 rounds of PCR. The PCR product is then cut with the restriction enzymes KpnI and EcoRV and ligated into the vector pcDNA3.1/Neo (+) (Invitrogen; Carlsbad, Calif.), which is also cut with the same restriction enzymes. The rest of the procedure for isolating plasmids containing the p40 gene, purifying endotoxin-free plasmid for transfection, transfection of CHO cells, and isolation of CHO cell clones expressing high amounts of human IL-12 p40 subunit is carried out in a manner identical to that described for the mouse clones. The two clones expressing the greatest amounts of p40 are then used in subsequent transfections with the p35 clone.

To express human IL-12 p35 cDNA from human cells is generated as described above for the p40 subunit. However, the restriction enzyme sites for NheI and KpnI are incorporated instead into the 5′- and 3′-ends of the PCR product respectively during the PCR reactions. The primers for the p35 sense strand are 5′-CCGCTAGCATGTGGCCCCCTGGGTCA -3′ (SEQ ID NO:13) and the primer sequence for the antisense strand is 5′-CCGGTACCGGAAGCATTCAGATAGCT-3′ (SEQ ID NO:14). The product is amplified by 35 rounds of PCR. The product is cut with the restriction enzymes NheI and KpnI and ligated into the vector pcDNA3.1/Hygro(+) (Invitrogen). Plasmid containing the p35 insert is also generated as described for the mouse.

For the ligation of the RGD-4C homing peptide to the human p35 subunit, two primers encoding the forward and reverse DNA sequences are synthesized (Operon Technologies; Alameda, Calif.). The sequence for the sense primer is identical to that used for the mouse system except different restriction sites are used, and the sequence for the antisense primer is complementary. Specifically, a KpnI site is incorporated into the 5′-end of the homing peptide DNA sequence and a XhoI site is incorporated into the 3′-end of the sequence. The primers are annealed as described for the mouse. The plasmid containing the p35 sequence (p35pcDNA3.1/Hygro (+)) is cut with the restriction enzymes KpnI and XhoI. The annealed oligonucleotides are also cut with these same enzymes. The products are then gel purified and ligated. Transformation of JM109 cells, purifying endotoxin-free plasmid for transfection, transfection of CHO cells, and isolation of CHO cell clones expressing high amounts of human IL-12 is carried out in a manner identical to that described for the mouse clones. Concentrations of human recombinant IL-12 (hrIL-12) can be determined via ELISA.

A CHO expression system for canine IL-12 can be produced in a manner identical to that described above for the human system with the following exceptions: the restriction site on the antisense primer must be changed from EcoRV to EcoRI, and the p40 subunit is ligated into the vector pcDNA3.1/myc-HisA. The change in restriction sites is necessary because the canine gene sequence contains an EcoRV restriction site, and this would result in cutting the gene in two. The canine p40 gene sequence does not contain an EcoRI site. The second change results in incorporation of a Histidine tag (His) onto the 5′-end of the p40 subunit of canine IL-12. This is necessary because there is currently no commercially available ELISA kit that can be used to detect the canine cytokine. However, an antibody for IL-12 is commercially available (R&D systems) for recognition of the canine protein (Helfand, S. C. Cancer res. 1999 59:3119-3127). This antibody can be used as the capture antibody in an ELISA system, and an antibody that recognizes the His-tag on the p40 subunit can be used as the detection antibody.

The primers used to amplify the canine p40 and p35 sequences are as follows:

p40 sense: 5′-CCGGTACCATGCATCCTCAGCAGTTG-3′ (SEQ ID NO:15)

p40 antisense: 5′-CCGAATTCACTGCAGGACACAGATGC-3′ (SEQ ID NO:16)

p35 sense: 5′-CCGCTAGCATGTGCCCGCCGCGCGGC-3′ (SEQ ID NO:17)

p35 antisense: 5′-CCGGTACCGGAAGAATTCAGATAACT-3′ (SEQ ID NO:18)

Fusion proteins comprising a shorter peptide containing only RGD can also be constructed. For these fusion proteins, the cloning and expression of the p40 subunit is identical to that described previously for each species. However, to incorporate RGD into, for example, the C-terminal end of the p35 subunit in any species, the nucleotide sequence for RGD is incorporated into each of the antisense primers described above. The primers also contain the proper restriction enzyme sites. The final product can be amplified by 35 rounds of PCR and ligated, cloned, selected, and expressed as described herein.

Further, as will be understood by those of skill in the art upon reading this disclosure, other methods than exemplified herein for production of plasmids comprising nucleic acid sequences for a mammalian IL-12 or biologically active fragments or variants thereof and methods for ligating a nucleic acid sequence encoding an RGD containing peptide to the plasmid comprising the mammalian IL-12 nucleic acid sequence, as well as other vectors and host cells for expression of the bifunctional fusion proteins, can be used. The presence of the IL-12 nucleic acids in the plasmid and DNA encoding the RGD containing peptide following ligation to a plasmid comprising a mammalian IL-12 nucleic acid sequence can be verified by routine sequencing. Preferred host cells for expression of the fusion protein are mammalian cells expressing only low levels or no αᵥβ₃. Examples include, but are not limited to, CHO cells and Saos-2 cells.

mrIL-12vp was produced from Chinese hamster ovary (CHO) cells transfected with plasmids containing p35RGD-4C and p40-His. Two transfected CHO clones, C2-A4 and C2-B3 were found to produce μg quantities of the fusion protein/10⁶ cells over a period of 24 hours.

IL-12 is a potent inducer of IFN-γ by T cells and Natural killer (NK) cells. Accordingly, the ability of the mrIL-12vp fusion protein to maintain biological activity of IL-12 was demonstrated using production of interferon-γ (IFN-γ) by stimulated murine splenocytes as a readout. Production of IFN-γ in cells stimulated with mrIL-12 was only slightly higher than cells stimulated with mrIL-12vp from either C2-A4 or C2-B3 cells, but this difference was not found to be statistically significant.

In addition, acute toxicity studies conducted in vivo in mice with the fusion protein showed no toxicity, thus indicating that toxicity of mrIL-12vp is not a limiting factor of these new therapeutic agents.

The ability of mrIL-12vp to inhibit neovessel growth was also compared with that of mrIL-12 in a corneal pocket assay. In these experiments, angiogenesis was induced using human bFGF (100 ng) and pumps containing mrIL-12vp, mrIL-12, phosphate buffered saline (PBS) or conditioned medium were placed subcutaneously into Balb/c mice. Examination of the corneas after 7 days of treatment showed that mrIL-12vp inhibited vessel growth at the lowest dose tested (0.25 μg/mouse/day). In contrast, inhibition by mrIL-12 was only observed at the highest dose (1.0 μg/mouse/day). No inhibition of vessel growth was observed in control mice treated with PBS. Because the fusion protein used comprised an enriched fraction and not the purified form of the protein, CHO conditioned medium that had been prepared in the same manner as the fusion protein was also tested to verify that the activity observed was due to mrIL-12vp and not to another component produced by the CHO cells or present in the medium. No differences in vessels growth were observed between these mice and PBS control treated mice indicating that the activity observed was due to mrIL-12vp.

The effects of mrIL-12vp and LM609, a monoclonal antibody specific for integrin αᵥβ₃, alone and in combination, on the growth of M21 human melanoma cells were also assessed. M21 cells are positive for the integrin αᵥβ₃, a surface protein that has been shown to bind ligands containing RGD. Using uptake of ³H-thymidine as a readout of cell proliferation, the growth of M21 cells was inhibited by mrIL-12vp at 24 hours, but not by IL-12 or LM609. The addition of LM609 appeared to block the activity of mrIL-12vp when the two were added in combination since the amount of growth inhibition observed was intermediate between the inhibition produced by mrIL-12vp alone and the lack of inhibition produced by LM609. The addition of mrIL-12 had no effect on the growth of the cells. To verify that mrIL-12vp is inhibiting the growth of cells through αᵥβ₃, experiments are also being conducted with cells that do not express the receptor, notably Saos-2 cells (human osteosarcoma) and M21-L cells, a derivative of the parental M21 line.

The ability of mrIL-12vp as compared to mrIL-12 to target M21 cells was also examined. In these experiments, M21 cells were allowed to adhere to a glass slide. Cells were then incubated with mrIL-12vp or mrIL-12. Binding of ligand to the cells was detected using a rabbit polyclonal antibody that recognizes the p40 subunit of murine IL-12, followed by a goat anti-rabbit antibody linked to FITC. Fluorescence of M21 cells was readily apparent in the cells incubated with mrIL-12vp, but not in the cells incubated with mrIL-12, indicating the specific delivery of IL-12 to tumor cells by mrIL-12vp. As a control, Saos-2 cells, which do not express αᵥβ₃, were used to verify the specificity of targeting. There was a lack of fluorescence of Saos-2 cells that had been incubated with mrIL-12vp or mrIL-12, indicating that the mrIL-12vp was binding specifically to the integrin αᵥβ₃. Incubation with secondary antibody alone was also used as a control to determine the amount of nonspecific binding by this antibody, which was found to be low.

Thus, as demonstrated herein, fusion proteins of the present invention comprising a mammalian IL-12 operably linked to a RGD-containing peptide provide a useful means for targeting IL-12 to tumor cells. As also demonstrated herein, the fusion proteins of the present invention inhibit angiogenesis as well as tumor cell growth in established in vitro and in vivo models. Accordingly, the fusion proteins of the present invention provide a useful means for treating tumor vaculature and tumors expressing αᵥβ₃ via IL-12 and for lowering the toxicity associated with IL-12 in mammals. For purposes of the present invention, by “mammal” it is meant to be inclusive, but not limited to, humans. Dosing regimes for the fusion proteins of the present invention can be routinely determined in accordance with pharmacological activity data from experiments in in vitro models such as described herein as well as previously established dosing regimes for IL-12. In a preferred embodiment, the fusion protein is diluted and delivered in a saline solution via a subcutaneous pump. Intravenous administration can also be performed. Amounts to be administered are based on the IL-12 equivalent of the fusion protein.

The following nonlimiting examples are provided to further illustrate the present invention.

### EXAMPLES

### Example 1

Expression of mrIL-12vp by CHO Cells

CHO cells transfected with plasmids containing p35RGD-4C and p40-His were allowed to grow for 2 weeks in 60 mm tissue culture dishes in the presence of 300 μg/ml hygromycin and 200 μg/ml G418. Individual foci arising from single cells were then picked and the cells were placed into 12 well tissue culture dishes. Once cells had reached confluency, the supernatant was removed and replaced with fresh medium. After 24 hours, supernatants from each clone were tested for the presence of the mrIL-12vp protein using a commercially available ELISA kit for the murine p70 whole IL-12 protein (Pharmingen, San Diego, Calif.). Two clones, C2-A4 and C2-B3 were found to produce μg quantities of the fusion protein/10⁶ cells over a period of 24 hours.

### Example 2

Production of IFN-γ by Murine Splenocytes after Addition of mrIL-12vp

For production of IFN-γ, splenocytes from the spleens of 7-8 week old male and female FVB mice were incubated with mrIL-12 or mrIL-12vp in 1 ml cultures (5×10⁶ cells/ml) in 24-well tissue culture plates for 48 hours at 37° C. in accordance with the procedure described by Schoenaut et al. (J. Immunol. 1992 148:3433-3400). The concentration of mrIL-12vp in the tissue culture supernatants was determined using the IL-12 ELISA kit as described in Example 1. Supernatant from a 2 day culture of C2-A4 cells contained 1.9 μg/ml of fusion protein and a 2-day culture of C2-B3 cells contained 1.2 μg/ml of fusion protein. Both samples along with mrIL-12 (R&D Systems, Minneapolis, Minn.) were diluted to a final concentration of 1 ng/ml (IL-12 equivalent) for the assay. After 48 hours, the supernatants were harvested and cells removed by centrifugation. Samples were then tested for IFN-γ using a commercially available ELISA kit (Pharmingen, San Diego, Calif.).

### Example 3

Toxicity Studies in Mice

C2-A4 CHO cells were placed in serum-free medium (AIM V, Life Technologies, Grand Island, N.Y.) for 48 hours and the tissue culture supernatant was concentrated using a Centricon Plus-20 ultrafiltration device (Millipore, Bedford, Mass.). The concentration of fusion protein was measured using the IL-12 ELISA kit described in Example 1. The protein was filter sterilized and diluted to 2.5 μg IL-12 equivalent/ml in sterile PBS and 100 μl (0.25 μg IL-12 equivalent) was injected i.p. into 8-10 week old male 129/J mice. Recombinant murine IL-12 (R&D Systems) was diluted in sterile PBS and injected into control mice at the same IL-12 concentrations used in the fusion protein group. Three mice were used in each group. Additionally, CHO cells not expressing the fusion protein were also incubated in AIM V medium and this supernatant was concentrated in a manner identical to the procedure used to concentrate mrIL-12vp as described supra. The supernatant was diluted in a similar manner to the supernatant containing mrIL-12vp and injected i.p. to control for the effects of components within the tissue culture supernatant. Mice were observed for 10 days, and no detrimental effects were observed from the injection of mrIL-12, mrIL-12vp, or medium, indicating that the fusion protein does not appear to be more toxic than mrIL-12 when administered via this protocol.

### Example 4

Corneal Neovascularization Assay

Polyvinyl sponges preirradiated to 2000 Gy (¹⁵⁷Cs source) were cut into 0.4×0.4×0.2 mm pieces and saturated with 100 ng of human recombinant bFGF in 1 μl. The loaded sponges were air-dried, covered with a layer of 12% Hydron S, and then dried under vacuum. Adult Balb/c mice were anesthetized, and sponges were introduced into a surgically created pocket in an avascular area of one cornea. Mouse eyes were examined daily for neovascularization using an ophthalmic scope. Two days after implantation, osmotic pumps (Alzet) containing mrIL-12, mrIL-12vp, CHO conditioned medium, or PBS were placed subcutaneously. Delivery of fluid from the pumps was approximately 1 μl/hour. After 7 days, 200 μl of FITC-conjugated high molecular weight dextran (3,000,000 MW) was injected into the tail vein, and the animals were sacrificed after 5 minutes. The eye was enucleated and fixed for 5 minutes in 4% formaldehyde. The cornea with the adjacent limbus was dissected, rinsed in PBS, and mounted on a glass slide with 10% glycerol. Phase contrast and fluorescence microscopy were used to visualize the overall appearance of the cornea and the presence of perfused blood vessels, respectively. Pumps were also removed from the mice and the volume of the remaining fluid was checked to insure that the delivery of the contents was consistent.

### Example 5

Growth Inhibition of M21 (Human Melanoma) Cells by mrIL-12vp

The effects of mrIL-12vp and anti-αᵥβ₃ monoclonal antibody LM609, alone and in combination on the growth of M21 cells (αᵥβ₃ positive ) were assessed after 24 hours in culture using ³H-thymidine as a readout. Cultures were performed in triplicate placing 100 μl of M21 cell suspension containing 5×10⁶ cells/ml in R-10 medium into flat-bottom microtiter wells for 2-4 hours in a humidified CO₂ incubator. After this incubation, mrIL-12, IL-12vp, or LM609 was added to the wells. Eight hours before termination of cultures, 0.5 μCi ³H-thymidine in 50 μl was added to individual wells. Cells were harvested on a Packard Filtermate Cell Harvester and counted on a Packard TopCount Microplate Scintillation Counter.

### Example 6

Immunofluorescence Labeling

M21 or Saos-2 cells were plated at 25,000 cells/well onto glass chamber slides. The cells were allowed to grow on the slides for 3 days or until optimal density was reached. The medium was then removed from the cells and the cells washed once in adhesion buffer, and 1 ml of adhesion buffer was added to each well. mrIL-12 (Peprotech) or mrIL-12vp was added to a final concentration of 2.5 μg IL-12 equivalent/ml and the slides were incubated for 30 min at 37° C. in a CO₂ incubator. At the end of the incubation period, slides were washed 3× for 5 minutes with PBS and then incubated for 15 min in 4% paraformaldehyde. Slides were washed 3× in PBS and then incubated for 10 minutes in ice-cold methanol followed by washing 3× with PBS. Slides were then blocked with goat serum for 1 hour at room temperature. Primary antibody against the mouse p40 IL-12 subunit was then added in a 1:500 dilution. Slides were incubated with the antibody for 2 hours at 4° C. After 2 hours, slides were washed 3× in PBS and incubated with goat anti-rabbit secondary antibody (1:250) linked to FITC for 1 hour at 4° C. Following this incubation, cells were washed 3× with PBS. An antifading agent, Vectashield (Vector) was added to the slides and a coverslip placed on each slide. Slide were then viewed under a fluorescence microscope and the images were recorded digitally.

What is claimed is:

1. A fusion protein comprising a mammalian interleukin-12 operably linked to a RGD-containing peptide.

2. The fusion protein of claim 1 wherein the RGD-containing peptide comprises SEQ ID NO:2.

3. The fusion protein of claim 1 wherein the RGD-containing peptide consists of RGD.

4. A nucleic acid sequence encoding the fusion protein of claim 1.

5. A vector comprising the nucleic acid sequence of claim 4.

6. A host cell expressing the vector of claim 5.

7. A method for inhibiting growth of angiogenic endothelial cells and αvβ3 positive tumor cells comprising targeting the endothelial cells and tumor cells with the fusion protein of claim 1.

8. A method for decreasing toxic side effects associated with interleukin-12 administration in a mammal comprising operatively linking interleukin-12 to a RGD-containing peptide via expression of a fusion protein comprising interleukin-12 and a RGD-containing peptide and administering to the mammal the fusion protein.

9. A method for treating cancer in a mammal comprising administering to the mammal a fusion protein comprising interleukin-12 operably linked to an RGD-containing peptide.


SEQUENCE LISTING (18 sequences)

SEQ ID NO: 1 - protein, 11 aa, Artificial Sequence
  Description of Artificial Sequence Synthetic
ACDCRGDCFCG

SEQ ID NO: 2 - protein, 9 aa, Artificial Sequence
  Description of Artificial Sequence Synthetic
CDCRGDCFC

SEQ ID NO: 3 - DNA, 27 bp, Artificial Sequence
  Description of Artificial Sequence Synthetic
ccggtaccatgtgtcaatcacgtctac

SEQ ID NO: 4 - DNA, 26 bp, Artificial Sequence
  Description of Artificial Sequence Synthetic
ccgatatctcaggcggagctcagata

SEQ ID NO: 5 - DNA, 49 bp, Artificial Sequence
  Description of Artificial Sequence Synthetic
ccggggagctctgtgactgtcgaggcgactgtttttgttaagatatcgg

SEQ ID NO: 6 - DNA, 26 bp, Artificial Sequence
  Description of Artificial Sequence Synthetic
ccggtaccatgtgtcctcagaagcta

SEQ ID NO: 7 - DNA, 26 bp, Artificial Sequence
  Description of Artificial Sequence Synthetic
ccgatatcctaggatcggaccctgca

SEQ ID NO: 8 - protein, 18 aa, Artificial Sequence
  Description of Artificial Sequence Synthetic
MSYLNASGTCDCRGDCFC

SEQ ID NO: 9 - protein, 18 aa, Artificial Sequence
  Description of Artificial Sequence Synthetic
MSYLNSSGTCDCRGDCFC

SEQ ID NO: 10 - protein, 16 aa, Artificial Sequence
  Description of Artificial Sequence Synthetic
MGYLSSACDCRGDCFC

SEQ ID NO: 11 - DNA, 26 bp, Artificial Sequence
  Description of Artificial Sequence Synthetic
ccggtaccatgtgtcaccagcagttg

SEQ ID NO: 12 - DNA, 26 bp, Artificial Sequence
  Description of Artificial Sequence Synthetic
ccgatatcctaactgcagggcacaga

SEQ ID NO: 13 - DNA, 26 bp, Artificial Sequence
  Description of Artificial Sequence Synthetic
ccgctagcatgtggccccctgggtca

SEQ ID NO: 14 - DNA, 26 bp, Artificial Sequence
  Description of Artificial Sequence Synthetic
ccggtaccggaagcattcagatagct

SEQ ID NO: 15 - DNA, 26 bp, Artificial Sequence
  Description of Artificial Sequence Synthetic
ccggtaccatgcatcctcagcagttg

SEQ ID NO: 16 - DNA, 26 bp, Artificial Sequence
  Description of Artificial Sequence Synthetic
ccgaattcactgcaggacacagatgc

SEQ ID NO: 17 - DNA, 26 bp, Artificial Sequence
  Description of Artificial Sequence Synthetic
ccgctagcatgtgcccgccgcgcggc

SEQ ID NO: 18 - DNA, 26 bp, Artificial Sequence
  Description of Artificial Sequence Synthetic
ccggtaccggaagaattcagataact